(19) United States
(12) Patent Application Publication
(10) Pub. No.: US 2025/0146010 A1
(43) Pub. Date: May 8, 2025

(54) COMPOSITIONS AND METHODS FOR SOYBEAN PLANT TRANSFORMATION

(71) Applicant: INARI AGRICULTURE TECHNOLOGY, INC., Cambridge, MA (US)

(72) Inventors: TEJINDER KUMAR MALL, WEST LAFAYETTE, IN (US); CHRISTOPHER ALEXANDER BAGLEY, LAFAYETTE, IN (US); RHODA ARABA TAWIAH BREW-APPIAH, WEST LAFAYETTE, IN (US); LARISSA ARRAIS GUIMARAES, WEST LAFAYETTE, IN (US)

(21) Appl. No.: 18/867,924

(22) PCT Filed: Jul. 11, 2023
(86) PCT No.: PCT/US2023/069952

§ 371 Date: Nov. 21, 2024

Related U.S. Application Data
(60) Provisional application No. 63/368,197, filed on Jul. 12, 2022.

(51) Int. Cl. C12N 15/82; C07K 14/415; C12N 9/10; C12N 9/12

(52) U.S. Cl. CPC C12N 15/8267; C07K 14/415; C12N 9/1022; C12N 9/12; C12N 15/8205; C12N 15/8274; C07K 2319/00

(57) ABSTRACT

Chimeric GRF-GIF polypeptides that can be used to improve the capacity of plant cells to regenerate embryogenic plant tissues, plant organs, and whole plants are disclosed. Also disclosed are plant cells comprising the chimeric polypeptides and related methods for improving the capacity of the plant cells to regenerate embryogenic plant tissues, plant organs, and whole plants.

### CROSS-REFERENCE TO RELATED APPLICATIONS

This International Patent application claims the benefit of U. S. Provisional Patent Application 63/368,197, filed on 12 Jul. 2022, which is incorporated by reference in its entirety herein.

### INCORPORATION OF SEQUENCE LISTING

This application contains a Sequence Listing which has been submitted in XML format and is hereby incorporated by reference in its entirety. Said XML copy, created 10 Jul. 2023, is named “P13865WO00_SequenceListing.xml” and is 15,177 bytes in size.

### BACKGROUND

To overcome recalcitrance of soybean to transformation, certain morphogenetic regulatory proteins have been introduced into soybean plant cells. A need exists for additional reagents to expand the range of germplasm accessible for transformation.

### SUMMARY

Recombinant polynucleotides comprising a polynucleotide encoding a chimeric GRF4-GIF1 (cGRF4-GIF1) polypeptide, a chimeric dicot plant GRF4-GIF1 (cdpGRF4-dpGIF1) polypeptide, or a chimeric GmGRF4-GmGIF1 (cGmGRF4-GmGIF1) polypeptide which comprises from its amino terminus (N-terminus) to its carboxy terminus (C-terminus) a GmGF4 polypeptide comprising an amino acid sequence having at least 92%, 95%, 98%, or 99% sequence identity across the entire length of SEQ ID NO: 1 and a GmGIF1 polypeptide comprising an amino acid sequence having at least 85%, 90%, 95%, 98%, or 99% sequence identity across the entire length of SEQ ID NO: 2 are provided. Compositions, soybean plant cells, and bacterial cells comprising the recombinant polynucleotides are also provided. Methods of producing a regenerable plant structure, the method comprising: (i) introducing the recombinant polynucleotide into a soybean plant cell; and (ii) culturing the soybean plant cell to produce the regenerable plant structure are also provided.

### DETAILED DESCRIPTION

Unless otherwise stated, nucleic acid sequences in the text of this specification are given, when read from left to right, in the 5′ to 3′ direction. Nucleic acid sequences may be provided as DNA or as RNA, as specified; disclosure of one necessarily defines the other, as well as necessarily defines the exact complements, as is known to one of ordinary skill in the art. Where a term is provided in the singular, the inventors also contemplate embodiments described by the plural of that term.

As used herein, an “acetohydroxyacid synthase (AHAS) inhibitor” refers to compounds which inhibit non-resistant (e.g. wild type) plant (e.g., soybean) AHAS enzymes.

Acetohydroxyacid synthase (AHAS) is also referred to as acetolactate synthase (ALS). AHAS inhibitors thus include compounds sometimes referred to as ALS inhibitors.

The phrase “allelic variant” as used herein refers to a polynucleotide or polypeptide sequence variant that occurs in a different strain, variety, or isolate of a given organism.

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the phrase “embryo productivity” refers to an assigned number that reflects how many somatic embryos develop on each immature embryo that is embryogenic, wherein the assigned number is determined by a subjective somatic embryogenesis score from 0 to 4 and wherein a higher score indicates an increase in somatic embryo production.

As used herein, “heterologous” refers to a polynucleotide or peptide sequence located in, e.g., a genome or a vector, in a context other than that in which the sequence occurs in nature. For example, a promoter that is operably linked to a gene other than the gene that the promoter is operably linked to in nature is a heterologous promoter.

The phrase “improved plant cell regenerative potential” as used herein refers to the ability of a given plant cell to form a somatic embryo, embryogenic callus, a somatic meristem, organogenic callus, a shoot, or a shoot further comprising roots in comparison to a control plant cell.

As used herein, the terms “include,” “includes,” and “including” are to be construed as at least having the features to which they refer while not excluding any additional unspecified features.

As used herein, the term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell and includes reference to the transient provision of a nucleic acid or protein to the cell. Introduced includes reference to stable or transient transformation methods. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., nuclear chromosome, plasmid, plastid, chloroplast, or mitochondrial DNA), converted into an autonomous replicon, or transiently expressed (e.g., transfected mRNA).

As used herein, the term “plant” includes a whole plant and any descendant, cell, tissue, or part of a plant. The term “plant parts” include any part(s) of a plant, including, for example and without limitation: seed (including mature seed and immature seed); a plant cutting; a plant cell; a plant cell culture; or a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. Regenerable cells in a plant cell or tissue culture may be embryos, protoplasts, meristematic cells, callus, pollen, leaves, anthers, roots, root tips, flowers, or stalks. In contrast, some plant cells are not capable of being regenerated to produce plants and are referred to herein as “non-regenerable” plant cells.

As used herein, the phrase “plant cell” can refer either a plant cell having a plant cell wall or to a plant cell protoplast lacking a plant cell wall.

As used herein, the phrase “somatic embryo induction frequency” refers to the percentage of immature embryos producing somatic embryos.

A polynucleotide or polypeptide has a certain percent “sequence identity” to another polynucleotide or polypeptide, meaning that, when aligned, that percentage of bases or amino acids are the same, and in the same relative position, when comparing the two sequences. To determine sequence identity, sequences can be aligned using the methods and computer program BLAST, available over the world wide web at ncbi.nlm.nih.gov/BLAST. See, e.g., Altschul et al. (1990), J. Mol. Biol. 215:403-10. Another alignment algorithm is FASTA, available in the Genetics Computing Group (GCG) package, from Madison, Wisconsin, USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, California, USA. Of particular interest are alignment programs that permit gaps in the sequence. The Smith-Waterman is one type of algorithm that permits gaps in sequence alignments. See Meth. Mol. Biol. 70:173-187 (1997). Also, the GAP program using the Needleman and Wunsch alignment method can be utilized to align sequences. See/. Mol. Biol. 48:443-453 (1970).

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

Dicot plant cells and related systems, methods, and compositions that provide for improved dicot plant cell regenerative potential in comparison to control dicot plant cells are provided herein. In certain embodiments, improved plant cell regenerative potential is provided by introducing a polynucleotide encoding a chimeric GRF4-GIF1 (cGRF4-GIF1) polypeptide, a chimeric dicot plant GRF4-GIF1 (cdpGRF4-dpGIF1) polypeptide, or a chimeric cGmGRF4-GmGIF1 polypeptide in the plant cell. In certain embodiments, the chimeric GRF4-GIF1 (cGRF4-GIF1) polypeptides comprise fusions of GRF4 and GIF1 peptides of monocot plants, dicot plants, or variants thereof. Non-limiting examples of GRF4 polypeptides and GIF1 polypeptides of monocot plants, dicot plants, variants thereof, and chimeras thereof which can be encoded by the recombinant polynucleotides provided herein include wheat, rice, Arabidopsis, Vitis, Citrus, and other GRF4 peptides, GIF1 peptides, and chimeras thereof set forth in WO2021/007284, which is incorporated herein by reference in its entirety. Other non-limiting examples of GIF1 polypeptides of monocot plants, dicot plants, variants thereof, and chimeras thereof which can be encoded by the recombinant polynucleotides provided herein include GIF1 peptides, and chimeras thereof set forth in U.S. Pat. No. 10,822,612 and WO2021185358, which are each incorporated herein by reference in their entireties. Non-limiting examples of dicot plant GRF4 polypeptides, GIF1 polypeptides, and chimeras thereof used in the recombinant polynucleotides provided herein include Arabidopsis, Vitis, and Citrus GRF4 polypeptides, GIF1 polypeptides, and chimeras thereof set forth in WO2021/007284. In certain embodiments, GRF4 peptides of monocot plants, dicot plants, variants thereof, and chimeras thereof encoded by the recombinant polynucleotides provided herein can comprise a GRF4 amino acid sequence having 60%, 70%, 75%, 80%, 85%, or 90% to 91%, 91.5%, or 91.9% sequence identity across the entire length of SEQ ID NO: 1 and optionally comprise a QLQ domain (e.g., SEQ ID NO: 4) and a WRC domain (e.g., SEQ ID NO: 5). Features of such QLQ domains include conserved Gln-Leu-Gln residues, conserved bulky aromatic/hydrophobic and acidic amino acid residues such as Phe, Trp, Tyr, Leu, Glu, or equivalents thereof, having similar hydrophobicity, polarity, or charge, and a conserved Pro residue (Kim J H, Choi D, Kende H. Plant J. 36, 94-104, (2003)). Features of such WRC (Trp, Arg, Cys) domains include the presence of multiple basic amino acids (Arg and Lys) and a C3H motif having conserved spacing of three Cys and one His residues (Kim J H, Choi D, Kende H. Plant J. 36, 94-104, (2003)). Without seeking to be limited by theory, the basic residues of the WRC domain are believed to serve as a nuclear localization signal while the C3H motif is believed to mediate binding to DNA and thus be involved in GRF polypeptide transcription factor activity. Non-limiting examples of QLQ domains include SEQ ID NO: 4 and QLQ domains disclosed in WO2021/007284. Non-limiting examples of WRC domains include SEQ ID NO: 5 and WRC domains disclosed in WO2021/007284. In certain embodiments, GIF1 polypeptides of monocot plants, dicot plants, variants thereof, and chimeras thereof encoded by the recombinant polynucleotides provided herein can comprise an amino acid sequence having 60%, 70%, or 75% to 80%, 82%, 84%, or 84.9% sequence identity across the entire length of SEQ ID NO: 2 and optionally comprise an SNH domain. Non-limiting examples of SNH domains include SEQ ID NO: 6 and SNH domains disclosed in WO2021/007284. Without seeking to be limited by theory, the SNH domain is believed to be involved in activity of the GIF protein as a transcription coactivator (Kim J H, Kende H (2004) Proc Natl Acad Sci USA 101:13374-13379). In certain embodiments, the polynucleotide encodes a cGmGRF4-GmGIF1 polypeptide comprising an amino acid sequence having at least 90%, at least 95%, at least 96%, at least 97%, at least 98%, at least 99%, at least 99.5%, or 100% sequence identity across the entire length of SEQ ID NO: 3. In certain embodiments, the cGmGRF4-GmGIF1 polypeptide can comprise a GmGRF4 polypeptide having at least 90%, at least 95%, at least 96%, at least 97%, at least 98%, at least 99%, or at least 99.5% sequence identity across the entire length of SEQ ID NO: 1 and optionally comprise a QLQ domain (e.g., SEQ ID NO: 4) and a WRC domain (e.g., SEQ ID NO: 5). In certain embodiments, the chimeric GRF4-GIF1 (cGRF4-GIF1) polypeptide, a chimeric dicot plant GRF4-GIF1 (cdpGRF4-dpGIF1) polypeptide, or a chimeric cGmGRF4-GmGIF1 will comprise a GRF4 polypeptide comprising a QLQ domain (e.g., SEQ ID NO: 4) and a WRC domain (e.g., SEQ ID NO: 5). In certain embodiments, the cGmGRF4-GmGIF1 polypeptide can comprise a GIF1 polypeptide having at least 90%, at least 95%, at least 96%, at least 97%, at least 98%, at least 99%, or at least 99.5% sequence identity across the entire length of SEQ ID NO: 2 and optionally comprise an SNH domain (e.g., SEQ ID NO: 6). In certain embodiments, the chimeric GRF4-GIF1 (cGRF4-GIF1) polypeptide, a chimeric dicot plant GRF4-GIF1 (cdpGRF4-dpGIF1) polypeptide, or the chimeric cGmGRF4-GmGIF1 will comprise a GIF1 polypeptide comprising an SNH domain (e.g., SEQ ID NO: 6). In certain embodiments, the cGRF4-GIF1, cdpGRF4-dpGIF1, or cGmGRF4-GmGIF1 polypeptides, homologs thereof, or variants thereof provided herein can significantly increase somatic embryogenesis from callus of otherwise recalcitrant germplasm (e.g., soybean germplasm).

In some embodiments, a chimeric GRF4-GIF1 (cGRF4-GIF1) polypeptide, a chimeric dicot plant GRF4-GIF1 (cdpGRF4-dpGIF1) polypeptide, or a cGmGRF4-GmGIF1 polypeptide can comprise a GRF4 polypeptide fused (e.g., covalently linked via peptide bonds) to a GIF1 peptide via a spacer polypeptide. Suitable spacer polypeptides include polypeptides of between 4 amino acids and 40 amino acids in length, or between 4 amino acids and 25 amino acids in length. These spacers can be produced by using synthetic, linker-encoding oligopeptides to couple the GRF4 and GIF1 proteins or can be encoded by a nucleic acid sequence encoding the cGRF4-GIF1, cdpGRF4-dpGIF1, or cGmGRF4-GmGIF1 polypeptide. Peptide spacers with a degree of flexibility can be used. The linking peptides may have virtually any amino acid sequence, bearing in mind that the preferred spacers will have a sequence that results in a generally flexible peptide. The use of small amino acids, such as glycine and alanine, are of use in creating a flexible peptide. A variety of different spacers are commercially available and are considered suitable for use. Examples of spacer polypeptides include glycine polymers (G) n, glycine-serine polymers (including, for example, (GS)n, GSGGSn (SEQ ID NO: 7), GGSGGSn (SEQ ID NO: 8), and GGGSn (SEQ ID NO: 9), where n is an integer of at least one), glycine-alanine polymers, alanine-serine polymers. Exemplary spacers can comprise amino acid sequences including, but not limited to, GGSG (SEQ ID NO: 10), GGSGG (SEQ ID NO: 11), GSGSG (SEQ ID NO: 12), GSGGG (SEQ ID NO: 13), GGGSG (SEQ ID NO: 14), GSSSG (SEQ ID NO: 15), and the like. In certain embodiments, the design of a peptide conjugated to any desired element can include spacers that are all or partially flexible, such that the spacer can include a flexible spacer as well as one or more portions that confer less flexible structure.

In certain embodiments, the polynucleotide encoding the cGRF4-GIF1, cdpGRF4-dpGIF1, or cGmGRF4-GmGIF1 polypeptide is introduced in isolated plant cells or plant protoplasts (i.e., are not located in undissociated or intact plant tissues, plant parts, or whole plants). In certain embodiments, the plant cells are obtained from any plant part or tissue or callus. In certain embodiments, the culture includes plant cells obtained from a plant tissue, a cultured plant tissue explant, whole plant, intact nodal bud, shoot apex or shoot apical meristem, root apex or root apical meristem, lateral meristem, intercalary meristem, seedling, whole seed, halved seed or other seed fragment, zygotic embryo, somatic embryo, immature embryo, ovule, pollen, microspore, anther, hypocotyl, cotyledon, leaf, petiole, stem, tuber, root, callus, or plant cell suspension. In certain embodiments, the plant cell is derived from a half or quarter embryo explant (e.g., soybean or another legume).

In certain embodiments, the polynucleotide encoding the cGRF4-GIF1, cdpGRF4-dpGIF1, or cGmGRF4-GmGIF1 polypeptide is introduced in plant cells that are located in undissociated or intact plant tissues, plant parts, plant explants, or whole plants. In certain embodiments, the plant cell can be located in an intact nodal bud, a cultured plant tissue explant, shoot apex or shoot apical meristem, root apex or root apical meristem, lateral meristem, intercalary meristem, seedling, whole seed, halved seed or other seed fragment, zygotic embryo, somatic embryo, immature embryo, ovule, pollen, microspore, anther, hypocotyl, cotyledon, leaf, petiole, stem, tuber, root, or callus. In certain embodiments, the explants used include immature embryos (e.g., immature soybean embryos). In certain embodiments, a quarter-seed meristem (“QSM”) explant including the whole embryo of a source seed, such as a leguminous seed (e.g., a soybean seed), wherein one-quarter of the cotyledon is left attached to the whole embryo, and wherein the shoot apical meristem tissue of the whole embryo is intact and is exposed by removal of the primary leaves, and wherein the radicle tip is trimmed is used (e.g., as set forth in U.S. Pat. No. 11,453,885, incorporated herein by reference in its entirety). In certain embodiments, a half-seed soybean explant is used (e.g., as set forth in U.S. Pat. No. 7,473,822, incorporated herein by reference in its entirety). In certain embodiments, an explant containing the plant cells (e.g., a QSM or half-seed explant) is optionally wounded, e.g., by creating incisions, punctures, or abrasions (e.g., by contacting the explant with a dry abrasive or a suspension of abrasive particulates, microparticulates, or nanoparticulates) on the explant's surface. In embodiments, the radicle tip is trimmed, e.g., with a scalpel. In certain embodiments, the aforementioned plant cells, meristems, or explants are obtained from a soybean (Glycine max)

In certain embodiments, the plant cells where the polynucleotide encoding the cGRF4-GIF1, cdpGRF4-dpGIF1, or cGmGRF4-GmGIF1 polypeptide is introduced, as well as the related methods, systems, or compositions provided herein can include plant cells obtained from or located in any dicot plant species of interest, for example, row crop or forage plants. In certain non-limiting embodiments, the plant cells are obtained from or located in soybean (Glycine max) or another legume (e.g., peas, beans, lentils, soybean, and forage legumes such as alfalfa or clover). Plant cells where the polynucleotide encoding the cGRF4-GIF1, cdpGRF4-dpGIF1, or cGmGRF4-GmGIF1 polypeptide is introduced include Phaseolus spp., Vigna spp., Vicia spp., Pisum spp., Lens spp., Medicago spp., and Trifolium spp. Non-limiting examples of plants suited to the use in the methods and systems disclosed herein also include Vigna spp., such as cowpea or black-eyed pea (Vigna unguiculate), adzuki bean (Vigna angularis), black gram (Vigna mungo), mung bean (Vigna radiata), and groundnut (Vigna subterranea), Cajanus spp., such as pigeon pea (Cajanus cajan), chickpea (Cicer arietinum), and common bean (Phaseolus spp.) varieties.

In certain embodiments, the plant cells can comprise haploid, diploid, or polyploid plant cells or plant protoplasts, for example, those obtained from a haploid, diploid, or polyploid plant, plant part or tissue, or callus. In certain embodiments, plant cells in culture (or the regenerated plant, progeny seed, and progeny plant) are haploid or can be induced to become haploid; techniques for making and using haploid plants and plant cells are known in the art, see, e.g., methods for generating haploids in Arabidopsis thaliana by crossing of a wild-type strain to a haploid-inducing strain that expresses altered forms of the centromere-specific histone CENH3, as described by Maruthachalam and Chan in “How to make haploid Arabidopsis thaliana”, protocol available at www [dot]openwetware [dot]org/images/d/d3/Haploid_Arabidopsis_protocol [dot]pdf; (Ravi et al. (2014) Nature Communications, 5:5334, doi: 10.1038/ncomms6334). Examples of haploid cells include but are not limited to plant cells obtained from haploid plants and plant cells obtained from reproductive tissues, e.g., from flowers, developing flowers or flower buds, ovaries, ovules, megaspores, anthers, pollen, megagametophyte, and microspores. In certain embodiments where the plant cell or plant protoplast is haploid, the genetic complement can be doubled by chromosome doubling (e.g., by spontaneous chromosomal doubling by meiotic non-reduction, or by using a chromosome doubling agent such as colchicine, oryzalin, trifluralin, pronamide, nitrous oxide gas, anti-microtubule herbicides, anti-microtubule agents, and mitotic inhibitors) in the plant cell or plant protoplast to produce a doubled haploid plant cell or plant protoplast wherein the complement of genes or alleles is homozygous; yet other embodiments include regeneration of a doubled haploid plant from the doubled haploid plant cell or plant protoplast. Another embodiment is related to a hybrid plant having at least one parent plant that is a doubled haploid plant provided by this approach. Production of doubled haploid plants provides homozygosity in one generation, instead of requiring several generations of self-crossing to obtain homozygous plants. The use of doubled haploids is advantageous in any situation where there is a desire to establish genetic purity (i.e. homozygosity) in the least possible time. Doubled haploid production can be particularly advantageous in slow-growing plants, such as fruit and other trees, or for producing hybrid plants that are offspring of at least one doubled-haploid plant.

In certain embodiments, the plant cells where the polynucleotide encoding the cGRF4-GIF1, cdpGRF4-dpGIF1, or cGmGRF4-GmGIF1 polypeptide is introduced can be plant cells that are (a) encapsulated or enclosed in or attached to a polymer (e.g., pectin, agarose, or other polysaccharide) or other support (solid or semi-solid surfaces or matrices, or particles or nanoparticles); (b) encapsulated or enclosed in or attached to a vesicle or liposome or other fluid compartment; or (c) not encapsulated or enclosed or attached. In certain embodiments, the plant cells can be in liquid or suspension culture or cultured in or on semi-solid or solid media, or in a combination of liquid and solid or semi-solid media (e.g., plant cells or protoplasts cultured on solid medium with a liquid medium overlay, or plant cells or protoplasts attached to solid beads or a matrix and grown with a liquid medium). In certain embodiments, the plant cells are encapsulated in a polymer (e.g., pectin, agarose, or other polysaccharide) or other encapsulating material, enclosed in a vesicle or liposome, suspended in a mixed-phase medium (such as an emulsion or reverse emulsion), or embedded in or attached to a matrix or other solid support (e.g., beads or microbeads, membranes, or solid surfaces).

In a related embodiment, the disclosure provides arrangements of plant cells having improved plant cell regenerative potential in the systems, methods, and compositions described herein, such as arrangements of plant cells convenient for screening purposes or for high-throughput and/or multiplex transformation or genome editing procedure. In an embodiment, the disclosure provides an arrangement of multiple plant cells comprising: (a) a polynucleotide encoding a cGRF4-GIF1, cdpGRF4-dpGIF1, or cGmGRF4-GmGIF1 polypeptide; and optionally (b) a genome editing system. In another embodiment, the disclosure provides an array including a plurality of containers, each including at least one plant cell or plant protoplast having improved plant cell regenerative potential. In an embodiment, the disclosure provides arrangements of plant cells having the polynucleotide encoding the cGRF4-GIF1, cdpGRF4-dpGIF1, or cGmGRF4-GmGIF1 polypeptide and optionally the genome editing system, wherein the plant cells are in an arrayed format, for example, in multi-well plates, encapsulated or enclosed in vesicles, liposomes, or droplets (useful, (e.g., in a microfluidics device), or attached discretely to a matrix or to discrete particles or beads; a specific embodiment is such an arrangement of multiple plant cells having improved plant cell regenerative potential provided in an arrayed format, further including a genome editing system (e.g., an RNA-guided nuclease, at least one guide RNA), which may be different for at least some locations on the array or even for each location on the array, and optionally at least one chemical, enzymatic, or physical delivery agent.

In the systems and methods provided herein, the polynucleotide encoding the cGRF4-GIF1, cdpGRF4-dpGIF1, or cGmGRF4-GmGIF1 polypeptide and a genome editing system can be introduced in the plant cell in any temporal order. In certain embodiments, the genome editing system and the polynucleotide encoding the cGRF4-GIF1, cdpGRF4-dpGIF1, or cGmGRF4-GmGIF1 polypeptide are introduced simultaneously. In other embodiments, the genome editing system is introduced after the polynucleotide encoding the cGRF4-GIF1, cdpGRF4-dpGIF1, or cGmGRF4-GmGIF1 polypeptide. In other embodiments, the genome editing system is introduced before the polynucleotide encoding the cGRF4-GIF1, cdpGRF4-dpGIF1, or cGRF4-GIF1, cdpGRF4-dpGIF1, or cGmGRF4-GmGIF1 polypeptide. In summary, the genome editing system can be provided to a plant cell either previous to, concurrently with, or subsequent to introducing the polynucleotide encoding the cGRF4-GIF1, cdpGRF4-dpGIF1, or cGmGRF4-GmGIF1 polypeptide to the plant cell.

Plant cells having improved plant cell regenerative potential conferred by expression of a cGRF4-GIF1, cdpGRF4-dpGIF1, or cGmGRF4-GmGIF1 polypeptide are provided herein. Also provided by the disclosure are compositions derived from or grown from the plant cell or plant protoplast having improved plant cell regenerative potential, provided by the systems and methods disclosed herein; such compositions include multiple protoplasts or cells, callus, a somatic embryo, a somatic meristem, embryogenic callus, or a regenerated plant grown from the plant cell or plant protoplast having improved plant cell regenerative potential. Improved plant cell regenerative potential in plant cells can be assessed by a variety of techniques. In certain embodiments, such techniques can compare the numbers and/or amount of regenerable plant structures (e.g., immature embryos, somatic embryos, embryogenic calli, somatic meristems, organogenic calli, shoots, or shoots further comprising roots) formed and/or recovered from a given number of plant cells comprising the polynucleotide encoding the cGRF4-GIF1, cdpGRF4-dpGIF1, or cGmGRF4-GmGIF1 polypeptide versus control plant cells without the polynucleotide. In certain embodiments, the regenerable structure is a shoot or a shoot which is selected for transfer to a rooting media (e.g., plant cell culture media comprising phytohormones which induce root formation). In certain embodiments, the methods provided herein can provide for increased transformation efficiencies (TE) when a regenerable shoot is transferred to rooting media at a shoot length of 1 cm to 1.5 cm, 2 cm, 3 cm or 4 cm. In certain embodiments, the methods provided herein can provide for increased transformation efficiencies (TE) when a regenerable shoot is transferred to rooting media at a shoot length of 1 cm to 2 cm or 1.3 to 1.8 cm. In certain embodiments, expression of the cGRF4-GIF1, cdpGRF4-dpGIF1, or cGmGRF4-GmGIF1 polypeptide results in an increased somatic embryo induction frequency or embryo productivity relative to a control plant cell lacking the polynucleotide encoding the polypeptide. In certain embodiments, the somatic embryo induction frequency is at least about 50%, at least about 60%, at least about 70%, at least about 80%, at least about 90%, or at least about 95%. In certain embodiments, somatic embryo induction frequency is increased at least 2-fold, at least 5-fold, at least 9-fold, at least 10-fold, or at least 20-fold relative to a control soybean plant cell lacking the polynucleotide encoding the polypeptide. In certain embodiments, somatic embryo induction frequency is increased by up to 2-fold, 5-fold, 9-fold, 10-fold, or 20-fold relative to a control soybean plant cell lacking the polynucleotide encoding the polypeptide. In certain embodiments, the embryo productivity is at least about 1, at least about 1.1, at least about 1.2, at least about 1.3, at least about 1.4, at least about 1.5, at least about 1.6, at least about 1.7, at least about 1.8, at least about 1.9, at least about 2, at least about 2.1, at least about 2.2, at least about 2.3, at least about 2.4, or at least about 2.5. In certain embodiments, the embryo productivity is from about 1 to about 3 or from about 1.5 to about 2.5. When both somatic embryo induction frequency and embryo productivity are high, the transformation rate is high and the culture is robust. In certain embodiments, expression of the cGRF4-GIF1, cdpGRF4-dpGIF1, or cGmGRF4-GmGIF1 polypeptide results in an increased percentage of transgenic or genome edited shoots recovered from the soybean plant cell in a transformation or genome editing procedure relative to a control plant cell lacking the polynucleotide encoding the polypeptide. In certain embodiments, the percentage of transgenic or genome edited shoots recovered from the soybean plant cell in a transformation or genome editing procedure is increased by up to 2-fold, 4-fold, 5-fold, 6-fold, 8-fold, 9-fold, 10-fold, or 12-fold in comparison to a control soybean plant cell lacking the chimeric polypeptide. In certain embodiments, the percentage of transgenic or genome edited shoots recovered from the soybean plant cell in a transformation or genome editing procedure is increased by up to 2-fold or 3-fold to 4-fold, 5-fold, 6-fold, 8-fold, 9-fold, 10-fold, or 12-fold in comparison to a control soybean plant cell lacking the chimeric polypeptide.

In certain embodiments, an attribute of tissues selected for introduction of the cGRF4-GIF1, cdpGRF4-dpGIF1, or cGmGRF4-GmGIF1 polynucleotides can be the presence of dividing cells and the ability to grow in tissue culture media. These tissues include, but are not limited to, dividing cells from seeds (e.g., embryos) and meristems of soybean plants and other aforementioned legumes. In certain embodiments, such increases in numbers and/or amounts of regenerable plant structures can be observed in about 1, 2, or 3 to about 7, 10, 14, 30, or 60 days following the introduction of a polynucleotide encoding the cGRF4-GIF1, cdpGRF4-dpGIF1, or cGmGRF4-GmGIF1 polypeptide. Methods for obtaining regenerable plant structures and regenerating plants from the plant cells, methods, and systems provided herein can be adapted from methods disclosed in U.S. Pat. No. 7,473,822 and US Patent Application Publication 20120156784, which are incorporated herein by reference in their entireties and specifically with respect to such disclosure. In certain embodiments, single plant cells subjected to the introduction of the polynucleotide will give rise to single regenerable plant structures. In certain embodiments, the single regenerable plant cell structure can form from a single cell on, or within, an explant that has been subjected to the introduction of the polynucleotide and optionally subjected to treatment with a genome editing system. In certain embodiments, initiation or formation of the single plant cell regenerable structure can occur where single-cell-derived cell or tissue proliferation (e.g., growth of callus, non-differentiated callus, embryogenic callus and organogenic callus) before initiation of the regenerable plant structure. In certain embodiments, regenerable plant structures from plant cells subjected to the introduction of the polynucleotide and optionally a genome editing system can be identified and/or selected via a positive growth selection based on the ability of those plant cells to initiate and/or form the regenerable plant structures more rapidly than adjacent plant cells that have not been subjected to the introduction of the polynucleotide. In certain embodiments, such positive growth selection can obviate or reduce the need to use a traditional negative selection system where an antibiotic or herbicide is used to inhibit growth of adjacent, non-transformed cells that do not contain a gene that confers resistance to the antibiotic or herbicide. Nonetheless, in certain embodiments, a selectable marker gene conferring resistance to an antibiotic, herbicide, or other agent can be introduced into the plant cell at least temporarily during initiation and/or formation of the regenerable plant cell structures to facilitate identification and recovery. In certain embodiments of the methods provided herein, a selectable marker gene conferring resistance of an acetohydroxyacid synthase (AHAS) inhibitor is used with an AHAS inhibitor. Selectable marker genes which confer resistance to AHAS inhibitors include: (i) modified SurA, SurB, Csr1, Csr1-1, and Csr1-2 genes (U.S. Pat. No. 11,371,055, incorporated herein by reference in its entirety); and (ii) various plant AHAS genes comprising one or more mutations which confer resistance to AHAS inhibitors (U.S. Pat. No. 11,198,883, incorporated herein by reference in its entirety). AHAS inhibitors include sulfonylurea, imidazolinone, triazoloyrimidine, or triazolinone herbicide. In certain embodiments, the sulfonylurea herbicide is bensulfuron-methyl, chlorsulfuron, ethametsulfuron-methyl, foramsulfuron, halosulfuron, mesosulfuron-methyl, metsulfuron-methyl, nicosulfuron, oxasulfuron, primisulfuron-methyl, prosulfuron, rimsulfuron, sulfometuron-methyl, sulfosulfuron, thifensulfuron-methyl, triasulfuron, or triflusulfuron-methyl. In certain embodiments, the imidazolinone herbicide is imazapyr, imazapic, imazethapyr, imazamox, imazamethabenz, or imazaquin.

In some embodiments, methods provided herein can include the additional step of growing or regenerating a plant from a plant cell comprising the polynucleotide encoding the cGRF4-GIF1, cdpGRF4-dpGIF1, or cGmGRF4-GmGIF1 polypeptide or from a regenerable plant structure obtained from that plant cell. In certain embodiments, the plant can further comprise an inserted transgene, a target gene edit, or genome edit as provided by the methods and compositions disclosed herein. In certain embodiments, callus is produced from the plant cell, and plantlets and plants produced from such callus. In other embodiments, whole seedlings or plants are grown directly from the plant cell without a callus stage. Thus, additional related aspects are directed to whole seedlings and plants grown or regenerated from the plant cell or plant protoplast having a target gene edit or genome edit, as well as the seeds of such plants. In certain embodiments wherein the plant cell or plant protoplast is subjected to genetic or epigenetic modification (for example, stable or transient expression of a transgene, gene silencing, epigenetic silencing, or genome editing by means of, e.g., an RNA-guided nuclease), the grown or regenerated plant exhibits a phenotype associated with the genetic or epigenetic modification. In certain embodiments, the grown or regenerated plant includes in its genome two or more genetic or epigenetic modifications that in combination provide at least one phenotype of interest. In certain embodiments, a heterogeneous population of plant cells having a target gene edit or genome edit, at least some of which include at least one genetic or epigenetic modification, is provided by the method; related aspects include a plant having a phenotype of interest associated with the genetic or epigenetic modification, provided by either regeneration of a plant having the phenotype of interest from a plant cell or plant protoplast selected from the heterogeneous population of plant cells having a target gene or genome edit, or by selection of a plant having the phenotype of interest from a heterogeneous population of plants grown or regenerated from the population of plant cells having a target gene edit or genome edit.

Examples of phenotypes of interest include herbicide resistance, improved tolerance of abiotic stress (e.g., tolerance of temperature extremes, drought, or salt) or biotic stress (e.g., resistance to nematode, bacterial, or fungal pathogens), improved utilization of nutrients or water, modified lipid, carbohydrate, or protein composition, improved flavor or appearance, improved storage characteristics (e.g., resistance to bruising, browning, or softening), increased yield, altered morphology (e.g., floral architecture or color, plant height, branching, root structure). In an embodiment, a heterogeneous population of plant cells having a target gene edit or genome edit (or seedlings or plants grown or regenerated therefrom) is exposed to conditions permitting expression of the phenotype of interest; e.g., selection for herbicide resistance can include exposing the population of plant cells having a target gene edit or genome edit (or seedlings or plants grown or regenerated therefrom) to an amount of herbicide or other substance that inhibits growth or is toxic, allowing identification and selection of those resistant plant cells (or seedlings or plants) that survive treatment. Methods for obtaining regenerable plant structures and regenerating plants from plant cells or regenerable plant structures can be adapted from published procedures (Paz et al., Plant Cell Reports, 25:206-213 (2006); Sato et al. Crop Sci. 44:646-652 (2004). Methods for obtaining regenerable plant structures and regenerating plants from plant cells or regenerable plant structures can also be adapted from U.S. Pat. No. 7,473,822 and US Patent Application Publication 20120156784, which are incorporated herein by reference in their entireties and specifically with respect to such disclosure. Also provided are heterogeneous populations, arrays, or libraries of such plants, succeeding generations or seeds of such plants grown or regenerated from the plant cells or plant protoplasts, having a target gene edit or genome edit, parts of the plants (including plant parts used in grafting as scions or rootstocks), or products (e.g., fruits or other edible plant parts, cleaned grains or seeds, edible oils, flours or starches, proteins, and other processed products) made from the plants or their seeds. Embodiments include plants grown or regenerated from the plant cells having a target gene edit or genome edit, wherein the plants contain cells or tissues that do not have a genetic or epigenetic modification, e.g., grafted plants in which the scion or rootstock contains a genetic or epigenetic modification, or chimeric plants in which some but not all cells or tissues contain a genetic or epigenetic modification. Grafted plants can be grafts between the same or different (generally related) species. Additional related aspects include a hybrid plant provided by crossing a first plant grown or regenerated from a plant cell or plant protoplast having a target gene edit or genome edit and having at least one genetic or epigenetic modification, with a second plant, wherein the hybrid plant contains the genetic or epigenetic modification; also contemplated is seed produced by the hybrid plant. Also envisioned as related aspects are progeny seed and progeny plants, including hybrid seed and hybrid plants, having the regenerated plant as a parent or ancestor. The plant cells and derivative plants and seeds disclosed herein can be used for various purposes useful to the consumer or grower. The intact plant itself may be desirable, e.g., plants grown as cover crops or as ornamentals. In other embodiments, processed products are made from the plant or its seeds, such as extracted proteins, oils, sugars, and starches, fermentation products, animal feed or human food, wood and wood products, pharmaceuticals, and various industrial products.

A cGRF4-GIF1, cdpGRF4-dpGIF1, or cGmGRF4-GmGIF1 polypeptide can be provided to a cell (e.g., a plant cell or plant protoplast) by any suitable technique. In certain embodiments, the cGRF4-GIF1, cdpGRF4-dpGIF1, or cGmGRF4-GmGIF1 polypeptide is provided by directly contacting a plant cell with the cGRF4-GIF1, cdpGRF4-dpGIF1, or cGmGRF4-GmGIF1 polypeptide or the polynucleotide that encodes the cGRF4-GIF1, cdpGRF4-dpGIF1, or cGmGRF4-GmGIF1 polypeptide. In certain embodiments, the cGRF4-GIF1, cdpGRF4-dpGIF1, or cGmGRF4-GmGIF1 polypeptide is provided by transporting the cGRF4-GIF1, cdpGRF4-dpGIF1, or cGmGRF4-GmGIF1 polypeptide or a polynucleotide that encodes cGRF4-GIF1, cdpGRF4-dpGIF1, or cGmGRF4-GmGIF1 polypeptide into a plant cell or plant protoplast using a chemical, enzymatic, or physical agent. In certain embodiments, the cGRF4-GIF1, cdpGRF4-dpGIF1, or cGmGRF4-GmGIF1 polypeptide is provided by bacterially mediated (e.g., Agrobacterium sp., Rhizobium sp., Sinorhizobium sp., Mesorhizobium sp., Bradyrhizobium sp., Azobacter sp., Phyllobacterium sp.) transfection of a plant cell or plant protoplast with a polynucleotide encoding the cGRF4-GIF1, cdpGRF4-dpGIF1, or cGmGRF4-GmGIF1 polypeptide; see, e.g., Broothaerts et al. (2005) Nature, 433:629-633. In an embodiment, the cGRF4-GIF1, cdpGRF4-dpGIF1, or cGmGRF4-GmGIF1 polypeptide is provided by transcription in a plant cell or plant protoplast of a DNA that encodes the polypeptide and is stably integrated in the genome of the plant cell or is provided to the plant cell or plant protoplast in the form of a plasmid or expression vector (e.g., a viral vector) that encodes the polypeptide. In certain embodiments, the polypeptide is provided to the plant cell or plant protoplast as a polynucleotide that encodes the polypeptide, e.g., in the form of an RNA (e.g., mRNA or RNA containing an internal ribosome entry site (IRES)) encoding the polypeptide. A genome editing system can also be introduced into the plant cells by similar techniques.

Transient expression of a cGRF4-GIF1, cdpGRF4-dpGIF1, or cGmGRF4-GmGIF1 polypeptide can be achieved by a variety of techniques. Certain embodiments are useful in effectuating transient expression of the cGRF4-GIF1, cdpGRF4-dpGIF1, or cGmGRF4-GmGIF1 polypeptide without remnants or selective genetic markers occurring in progeny. In certain embodiments, the cGRF4-GIF1, cdpGRF4-dpGIF1, or cGmGRF4-GmGIF1 polypeptide is provided directly to the plant cells, systems, methods, and compositions as isolated molecules, as isolated or semi-purified products of a cell free synthetic process (e.g., in vitro translation), or as isolated or semi-purified products of a cell-based synthetic process (e.g., such as in a bacterial or other cell lysate). In certain embodiments, the cGRF4-GIF1, cdpGRF4-dpGIF1, or cGmGRF4-GmGIF1 polypeptide is targeted to the plant cell or cell nucleus in a manner that ensures transient expression (e.g., by methods adapted from Gao et al. 2016; or Li et al. 2009). In certain embodiments, the cGRF4-GIF1, cdpGRF4-dpGIF1, or cGmGRF4-GmGIF1 polypeptide is delivered into the plant cell by delivery of the polypeptide itself in the absence of any polynucleotide that encodes the polypeptide. In certain embodiments, the cGRF4-GIF1, cdpGRF4-dpGIF1, or cGmGRF4-GmGIF1 protein can be produced in a heterologous system, purified and delivered to plant cells by particle bombardment (e.g., by methods adapted from Martin-Ortigosa and Wang; Transgenic Res. 2014 October; 23 (5): 743-56. doi: 10.1007/s11248-014-9807-y.). In embodiments where the cGRF4-GIF1, cdpGRF4-dpGIF1, or cGmGRF4-GmGIF1 polypeptide is delivered in the absence of any encoding polynucleotides, the delivered polypeptide is expected to degrade over time in the absence of ongoing expression from any introduced encoding polynucleotides to result in transient expression. In certain embodiments, the cGRF4-GIF1, cdpGRF4-dpGIF1, or cGmGRF4-GmGIF1 polypeptide is delivered into the plant cell by delivery of a polynucleotide that encodes the polypeptide. In certain embodiments, the polypeptide can be encoded on a bacterial plasmid and delivered to plant tissue by particle bombardment (e.g., by methods adapted from Hamada et al. 2018; or Kirienko, Luo, and Sylvester 2012). In certain embodiments, the polypeptide can be encoded on a T-DNA and transiently transferred to plant cells using Agrobacterium (e.g., by methods adapted from Leonelli et al. The Plant Journal (2016) 88, 375-386; or Wu et al. Plant Methods (2014) 10, 19).

In certain embodiments, the polypeptide can be encoded in a viral genome and delivered to plants (e.g., by methods adapted from Honig et al. 2015 Molecular Plant 8, 1292-1294). In certain embodiments, the polypeptide can be encoded in mRNA or an RNA comprising an IRES and delivered to target plant cells. In certain embodiments, the cGRF4-GIF1, cdpGRF4-dpGIF1, or cGmGRF4-GmGIF1 polypeptide is delivered into the plant cell by delivery of a polynucleotide that encodes the polypeptide. In certain embodiments, the polynucleotide that encodes the cGRF4-GIF1, cdpGRF4-dpGIF1, or cGmGRF4-GmGIF1 polypeptide is not integrated into a plant cell genome (e.g., as a polynucleotide lacking sequences that provide for integration, by agroinfiltration on an integration deficient T-DNA vector or system, or in a viral vector), is not operably linked to polynucleotides which provide for autonomous replication, and/or only provided with factors (e.g., viral replication proteins) that provide for autonomous replication. Suitable techniques for transient expression including biolistic and other delivery of polynucleotides, agroinfiltration, and use of viral vectors disclosed by Canto, 2016 and others can be adapted for transient expression of the polypeptides provided herein. In certain embodiments, the polynucleotide that encodes the cGRF4-GIF1, cdpGRF4-dpGIF1, or cGmGRF4-GmGIF1 polypeptide is integrated into a plant cell genome (e.g., a nuclear or plastid genome) and transient expression of the polypeptide is effectuated by excision of the polynucleotide and/or regulated expression of the polypeptide. Excision of a polynucleotide encoding the polypeptide can be provided by use of site-specific recombination systems (e.g., Cre-Lox, FLP-FRT). Regulated expression of the polypeptide can be effectuated by methods including: (i) operable linkage of the polynucleotide encoding the polypeptide to a developmentally-regulated, de-repressable, and/or inducible promoter; and/or (ii) introduction of a polynucleotide (e.g., dsRNA or amiRNA) that can induce siRNA-mediated inhibition of the polypeptide. Suitable site-specific recombination systems as well as developmentally-regulated, de-repressable, and/or inducible promoters include those disclosed in US Patent Application Publication No. 20170121722, which is incorporated herein by reference in its entirety and specifically with respect to such disclosure. Polynucleotides that can be used to effectuate transient expression of the cGRF4-GIF1, cdpGRF4-dpGIF1, or cGmGRF4-GmGIF1 polypeptide include: (a) double-stranded RNA; (b) single-stranded RNA; (c) chemically modified RNA; (d) double-stranded DNA; (e) single-stranded DNA; (f) chemically modified DNA; or (g) a combination of (a)-(f). Certain embodiments of the polynucleotide further include additional nucleotide sequences that provide useful functionality; non-limiting examples of such additional nucleotide sequences include an aptamer or riboswitch sequence, nucleotide sequence that provides secondary structure such as stem-loops or that provides a sequence-specific site for an enzyme (e.g., a sequence-specific recombinase or endonuclease site), T-DNA (e.g., DNA sequence encoding a cGRF4-GIF1, cdpGRF4-dpGIF1, or cGmGRF4-GmGIF1 polypeptide is enclosed between left and right T-DNA borders from Agrobacterium spp. or from other bacteria that infect or induce tumors in plants), a DNA nuclear-targeting sequence, a regulatory sequence such as a promoter sequence, and a transcript-stabilizing or -destabilizing sequence. Certain embodiments of the polynucleotide include those wherein the polynucleotide is complexed with, or covalently or non-covalently bound to, a non-nucleic acid element, e.g., a carrier molecule, an antibody, an antigen, a viral movement protein, a cell-penetrating or pore-forming peptide, a polymer, a detectable label, a quantum dot, or a particulate or nanoparticulate.

Transient expression of the cGRF4-GIF1, cdpGRF4-dpGIF1, or cGmGRF4-GmGIF1 polypeptide can be for a period of time and/or in an amount sufficient to result in improved regenerative potential in comparison to a control plant cell. In certain embodiments, the transient increase in the expression of the cGRF4-GIF1, cdpGRF4-dpGIF1, or cGmGRF4-GmGIF1 polypeptide is for a period of about 1, 2, 4, 8, 12, 16, 20, 24, 30, or 36 hours to about 72, 96, 120, 144, 168, 192, 276, or 336 hours. In certain embodiments, the transient increase in the expression of the cGRF4-GIF1, cdpGRF4-dpGIF1, or cGmGRF4-GmGIF1 polypeptide is for a period of about 2, 4, 8, 12, or 16 hours to about 18, 20, 24, 30 or 36 hours. In certain embodiments, the transient increase in the expression of the cGRF4-GIF1, cdpGRF4-dpGIF1, or cGmGRF4-GmGIF1 polypeptide is for a period of about 18, 20, 24, 30 or 36 hours to about 60, 80, 100, 120, 168, or 192 hours. Such transient increases in expression of cGRF4-GIF1, cdpGRF4-dpGIF1, or cGmGRF4-GmGIF1 polypeptide can be measured by methods whereby accumulated gene products including mRNAs and/or proteins are measured. Useful methods of measuring mRNAs include quantitative reverse transcriptase Polymerase Chain Reaction (qRT-PCR)-based and/or any hybridization-based assay. Useful methods for quantitating proteins include immunoassays (e.g., ELISAs, RIAs) and/or mass spectrometry-based methods.

Genome editing systems of use in the methods provided herein include molecules capable of introducing a double-strand break (“DSB”) or single-strand break (“SSB”) at a specific site or sequence in a double-stranded DNA, such as in genomic DNA or in a target gene located within the genomic DNA as well as accompanying guide RNA or donor or other DNA template polynucleotides. Examples of such gene editing molecules include: (a) a nuclease comprising an RNA-guided nuclease, an RNA-guided DNA endonuclease or RNA directed DNA endonuclease (RdDe), a class 1 CRISPR type nuclease system, a type II Cas nuclease, a Cas9, a nCas9 nickase, a type V Cas nuclease, a Cas12a nuclease, a nCas12a nickase, a Cas12d (CasY), a Cas12e (CasX), a Cas12b (C2c1), a Cas12c (C2c3), a Cas12i, a Cas12j, a Cas12L, a Cas14, an engineered nuclease, a codon-optimized nuclease, a zinc-finger nuclease (ZFN) or nickase, a transcription activator-like effector nuclease (TAL-effector nuclease or TALEN) or nickase (TALE-nickase), an Argonaute, and a meganuclease or engineered meganuclease; (b) a polynucleotide encoding one or more nucleases capable of effectuating site-specific alteration (including introduction of a DSB or SSB) of a target nucleotide sequence; (c) a guide RNA (gRNA) for use with an RNA-guided nuclease, or a DNA encoding a gRNA for use with an RNA-guided nuclease; (d) donor DNA template polynucleotides suitable for insertion at a break in genomic DNA by homology-directed repair (HDR) or microhomology-mediated end joining (MMEJ); and (e) other DNA templates (e.g., dsDNA, ssDNA, or combinations thereof) suitable for insertion at a break in genomic DNA (e.g., by non-homologous end joining (NHEJ).

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1. Plant RNA promoters for expressing CRISPR guide RNA and plant codon-optimized CRISPR Cas9 endonuclease are disclosed in International Patent Application PCT/US2015/018104 (published as WO 2015/131101 and claiming priority to U.S. Provisional Patent Application 61/945,700). Methods of using CRISPR technology for genome editing in plants are disclosed in US Patent Application Publications US 2015/0082478A1 and US 2015/0059010A1 and in International Patent Application PCT/US2015/038767 A1 (published as WO 2016/007347 and claiming priority to U.S. Provisional Patent Application 62/023,246). In certain embodiments, an RNA-guided endonuclease that leaves a blunt end following cleavage of the target site is used. Blunt-end cutting RNA-guided endonucleases include Cas9, Cas12c, Cas12i, and Cas 12h (Yan et al., 2019). In certain embodiments, an RNA-guided endonuclease that leaves a staggered single stranded DNA overhanging end following cleavage of the target site is used. Staggered-end cutting RNA-guided endonucleases include Cas12a, Cas12b, and Cas12e. All of the patent publications referenced in this paragraph are incorporated herein by reference in their entirety.

CRISPR-type genome editing can be adapted for use in the plant cells and methods provided herein in several ways. CRISPR elements, e.g., gene editing molecules comprising CRISPR endonucleases and CRISPR guide RNAs including single guide RNAs or guide RNAs in combination with tracrRNAs or scoutRNA, or polynucleotides encoding the same, are useful in effectuating genome editing without remnants of the CRISPR elements or selective genetic markers occurring in progeny. In certain embodiments, the CRISPR elements are provided directly to the eukaryotic cell (e.g., plant cells), systems, methods, and compositions as isolated molecules, as isolated or semi-purified products of a cell free synthetic process (e.g., in vitro translation), or as isolated or semi-purified products of in a cell-based synthetic process (e.g., such as in a bacterial or other cell lysate). In certain embodiments, plants or plant cells used in the systems, methods, and compositions provided herein can comprise a transgene that expresses a CRISPR endonuclease (e.g., a Cas9, a Cpf1-type or other CRISPR endonuclease). In certain embodiments, one or more CRISPR endonucleases with unique PAM recognition sites can be used. Guide RNAs (sgRNAs or crRNAs and a tracrRNA) form an RNA-guided endonuclease/guide RNA complex which can specifically bind sequences in the gDNA target site that are adjacent to a protospacer adjacent motif (PAM) sequence. The type of RNA-guided endonuclease typically informs the location of suitable PAM sites and design of crRNAs or sgRNAs. G-rich PAM sites, e.g., 5′-NGG are typically targeted for design of crRNAs or sgRNAs used with Cas9 proteins. Examples of PAM sequences include 5′-NGG (Streptococcus pyogenes), 5′-NNAGAA (Streptococcus thermophilus CRISPR1), 5′-NGGNG (Streptococcus thermophilus CRISPR3), 5′-NNGRRT or 5′-NNGRR (Staphylococcus aureus Cas9, SaCas9), and 5′-NNNGATT (Neisseria meningitidis). T-rich PAM sites (e.g., 5′-TTN or 5′-TTTV, where “V” is A, C, or G) are typically targeted for design of crRNAs or sgRNAs used with Cas12a proteins. In some instances, Cas12a can also recognize a 5′-CTA PAM motif. Other examples of potential Cas12a PAM sequences include TTN, CTN, TCN, CCN, TTTN, TCTN, TTCN, CTTN, ATTN, TCCN, TTGN, GTTN, CCCN, CCTN, TTAN, TCGN, CTCN, ACTN, GCTN, TCAN, GCCN, and CCGN (wherein N is defined as any nucleotide). Cpf1 endonuclease and corresponding guide RNAs and PAM sites are disclosed in US Patent Application Publication 2016/0208243 A1, which is incorporated herein by reference for its disclosure of DNA encoding Cpf1 endonucleases and guide RNAs and PAM sites.

For the purposes of gene editing, CRISPR arrays can be designed to contain one or multiple guide RNA sequences directed to a desired target DNA sequence; see, for example, Cong et al. (2013) Science, 339:819-823; Ran et al. (2013) Nature Protocols, 8:2281-2308. At least 16 or 17 nucleotides of gRNA sequence are required by Cas9 for DNA cleavage to occur; for Cpf1 at least 16 nucleotides of gRNA sequence are needed to achieve detectable DNA cleavage and at least 18 nucleotides of gRNA sequence were reported necessary for efficient DNA cleavage in vitro; see Zetsche et al. (2015) Cell, 163:759-771. In practice, guide RNA sequences are generally designed to have a length of 17-24 nucleotides (frequently 19, 20, or 21 nucleotides) and exact complementarity (i.e., perfect base-pairing) to the targeted gene or nucleic acid sequence; guide RNAs having less than 100% complementarity to the target sequence can be used (e.g., a gRNA with a length of 20 nucleotides and 1-4 mismatches to the target sequence) but can increase the potential for off-target effects. The design of effective guide RNAs for use in plant genome editing is disclosed in US Patent Application Publication 2015/0082478 A1, the entire specification of which is incorporated herein by reference. Efficient gene editing has been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing); see, for example, Cong et al. (2013) Science, 339:819-823; Xing et al. (2014) BMC Plant Biol., 14:327-340. Chemically modified sgRNAs have been demonstrated to be effective in genome editing; see, for example, Hendel et al. (2015) Nature Biotechnol., 985-991. The design of effective gRNAs for use in plant genome editing is disclosed in US Patent Application Publication 2015/0082478 A1, the entire specification of which is incorporated herein by reference.

Other nucleases capable of effecting site-specific modification of a target nucleotide sequence in the systems, methods, and compositions provided herein include zinc-finger nucleases (ZFNs), transcription activator-like effector nucleases (TAL-effector nucleases or TALENs), Argonaute proteins, and a meganuclease or engineered meganuclease. Zinc finger nucleases (ZFNs) are engineered proteins comprising a zinc finger DNA-binding domain fused to a nucleic acid cleavage domain, e.g., a nuclease. The zinc finger binding domains provide specificity and can be engineered to specifically recognize any desired target DNA sequence. For a review of the construction and use of ZFNs in plants and other organisms, see, e.g., Urnov et al. (2010) Nature Rev. Genet., 11:636-646. The zinc finger DNA binding domains are derived from the DNA-binding domain of a large class of eukaryotic transcription factors called zinc finger proteins (ZFPs). The DNA-binding domain of ZFPs typically contains a tandem array of at least three zinc “fingers” each recognizing a specific triplet of DNA. A number of strategies can be used to design the binding specificity of the zinc finger binding domain. One approach, termed “modular assembly”, relies on the functional autonomy of individual zinc fingers with DNA. In this approach, a given sequence is targeted by identifying zinc fingers for each component triplet in the sequence and linking them into a multifinger peptide. Several alternative strategies for designing zinc finger DNA binding domains have also been developed. These methods are designed to accommodate the ability of zinc fingers to contact neighboring fingers as well as nucleotide bases outside their target triplet. Typically, the engineered zinc finger DNA binding domain has a novel binding specificity, compared to a naturally-occurring zinc finger protein. Engineering methods include, for example, rational design and various types of selection. Rational design includes, for example, the use of databases of triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, e.g., U.S. Pat. Nos. 6,453,242 and 6,534,261, both incorporated herein by reference in their entirety. Exemplary selection methods (e.g., phage display and yeast two-hybrid systems) are well known and described in the literature. In addition, enhancement of binding specificity for zinc finger binding domains has been described in U.S. Pat. No. 6,794,136, incorporated herein by reference in its entirety. In addition, individual zinc finger domains may be linked together using any suitable linker sequences. Examples of linker sequences are publicly known, e.g., see U.S. Pat. Nos. 6,479,626; 6,903,185; and 7,153,949, incorporated herein by reference in their entirety. The nucleic acid cleavage domain is non-specific and is typically a restriction endonuclease, such as Fokl. This endonuclease must dimerize to cleave DNA. Thus, cleavage by Fokl as part of a ZFN requires two adjacent and independent binding events, which must occur in both the correct orientation and with appropriate spacing to permit dimer formation. The requirement for two DNA binding events enables more specific targeting of long and potentially unique recognition sites. Fokl variants with enhanced activities have been described; see, e.g., Guo et al. (2010) J. Mol. Biol., 400:96-107.

Transcription activator like effectors (TALEs) are proteins secreted by certain Xanthomonas species to modulate gene expression in host plants and to facilitate the colonization by and survival of the bacterium. TALEs act as transcription factors and modulate expression of resistance genes in the plants. Recent studies of TALEs have revealed the code linking the repetitive region of TALEs with their target DNA-binding sites. TALEs comprise a highly conserved and repetitive region consisting of tandem repeats of mostly 33 or 34 amino acid segments. The repeat monomers differ from each other mainly at amino acid positions 12 and 13. A strong correlation between unique pairs of amino acids at positions 12 and 13 and the corresponding nucleotide in the TALE-binding site has been found. The simple relationship between amino acid sequence and DNA recognition of the TALE binding domain allows for the design of DNA binding domains of any desired specificity. TALEs can be linked to a non-specific DNA cleavage domain to prepare genome editing proteins, referred to as TAL-effector nucleases or TALENs. As in the case of ZFNs, a restriction endonuclease, such as Fokl, can be conveniently used. For a description of the use of TALENs in plants, see Mahfouz et al. (2011) Proc. Natl. Acad. Sci. USA, 108:2623-2628 and Mahfouz (2011) GM Crops, 2:99-103.

Argonautes are proteins that can function as sequence-specific endonucleases by binding a polynucleotide (e.g., a single-stranded DNA or single-stranded RNA) that includes sequence complementary to a target nucleotide sequence) that guides the Argonaute to the target nucleotide sequence and effects site-specific alteration of the target nucleotide sequence; see, e.g., US Patent Application Publication 2015/0089681, incorporated herein by reference in its entirety.

In related embodiments, zinc finger nucleases, TALENs, and Argonautes are used in conjunction with other functional domains. For example, the nuclease activity of these nucleic acid targeting systems can be altered so that the enzyme binds to but does not cleave the DNA. Examples of functional domains include transposase domains, integrase domains, recombinase domains, resolvase domains, invertase domains, protease domains, DNA methyltransferase domains, DNA hydroxylmethylase domains, DNA demethylase domains, histone acetylase domains, histone deacetylase domains, nuclease domains, repressor domains, activator domains, nuclear-localization signal domains, transcription-regulatory protein (or transcription complex recruiting) domains, cellular uptake activity associated domains, nucleic acid binding domains, antibody presentation domains, histone modifying enzymes, recruiter of histone modifying enzymes; inhibitor of histone modifying enzymes, histone methyltransferases, histone demethylases, histone kinases, histone phosphatases, histone ribosylases, histone deribosylases, histone ubiquitinases, histone deubiquitinases, histone biotinases and histone tail proteases. Non-limiting examples of functional domains include a transcriptional activation domain, a transcription repression domain, and an SHH1, SUVH2, or SUVH9 polypeptide capable of reducing expression of a target nucleotide sequence via epigenetic modification; see, e.g., US Patent Application Publication 2016/0017348, incorporated herein by reference in its entirety. Genomic DNA may also be modified via base editing using a fusion between a catalytically inactive Cas9 (dCas9) fused to a cytidine deaminase which converts cytosine (C) to uridine (U), thereby effecting a C to T substitution; see Komor et al. (2016) Nature, 533:420-424. In other embodiments, adenine base editors (ABEs) can be used to convert A/T base pairs to G/C base pairs in genomic DNA (Gaudelli et al., 2017).

Other genome altering reagents used in plant cells and methods provided herein include transgenes or vectors comprising the same. Such transgenes can confer useful traits that include herbicide tolerance, pest tolerance (e.g., tolerance to insects, nematodes, or plant pathogenic fungi and bacteria), improved yield, increased and/or qualitatively improved oil, starch, and protein content, improved abiotic stress tolerance (e.g., improved or enhanced water use efficiency or drought tolerance, osmotic stress tolerance, high salinity stress tolerance, heat stress tolerance, enhanced cold tolerance, including cold germination tolerance), and the like. Such transgenes include both transgenes that confer the trait by expression of an exogenous protein as well as transgenes that confer the trait by inhibiting expression of endogenous plant genes (e.g., by inducing an siRNA response which inhibits expression of the endogenous plant genes). Transgenes that can provide such traits are disclosed in US Patent Application Publication Nos. 20170121722 and 20170275636, which are each incorporated herein by reference in their entireties and specifically with respect to such disclosures.

In some embodiments, one or more polynucleotides or vectors driving expression of one or more polynucleotides encoding any of the cGmGRF4-GmGIF1 polypeptides and/or genome editing systems are introduced into a plant cell. In certain embodiments, a polynucleotide vector comprises a regulatory element such as a promoter operably linked to one or more polynucleotides encoding the polypeptide or genome editing system. In such embodiments, expression of these polynucleotides can be controlled by selection of the appropriate promoter, particularly promoters functional in a plant cell; useful promoters include constitutive, conditional, inducible, and temporally or spatially specific promoters (e.g., a tissue specific promoter, a developmentally regulated promoter, or a cell cycle regulated promoter). Developmentally regulated promoters that can be used include Phospholipid Transfer Protein (PLTP), fructose-1,6-bisphosphatase protein, NAD (P)-binding Rossmann-Fold protein, adipocyte plasma membrane-associated protein-like protein, Rieske [2Fe-2S] iron-sulfur domain protein, chlororespiratory reduction 6 protein, D-glycerate 3-kinase, chloroplastic-like protein, chlorophyll a-b binding protein 7, chloroplastic-like protein, ultraviolet-B-repressible protein, Soul heme-binding family protein, Photosystem I reaction center subunit psi-N protein, and short-chain dehydrogenase/reductase protein that are disclosed in US Patent Application Publication No. 20170121722, which is incorporated herein by reference in its entirety and specifically with respect to such disclosure. In certain embodiments, the promoter is operably linked to nucleotide sequences encoding multiple guide RNAs, wherein the sequences encoding guide RNAs are separated by a cleavage site such as a nucleotide sequence encoding a microRNA recognition/cleavage site or a self-cleaving ribozyme (see, e.g., Ferré-D'Amaré and Scott (2014) Cold Spring Harbor Perspectives Biol., 2:a003574). In certain embodiments, the promoter is an RNA polymerase III promoter operably linked to a nucleotide sequence encoding one or more guide RNAs. In certain embodiments, the promoter operably linked to one or more polynucleotides is a constitutive promoter that drives gene expression in plant cells. In certain embodiments, the promoter drives gene expression in the nucleus or in an organelle such as a chloroplast or mitochondrion. Examples of constitutive promoters include a CaMV 35S promoter as disclosed in U.S. Pat. Nos. 5,858,742 and 5,322,938, a rice actin promoter as disclosed in U.S. Pat. No. 5,641,876, a maize chloroplast aldolase promoter as disclosed in U.S. Pat. No. 7,151,204, and the nopaline synthase (NOS) and octopine synthase (OCS) promoters from Agrobacterium tumefaciens. In certain embodiments, the promoter operably linked to one or more polynucleotides encoding elements of a genome-editing system is a promoter from figwort mosaic virus (FMV), a RUBISCO promoter, or a pyruvate phosphate dikinase (PPDK) promoter, which is active in photosynthetic tissues. Other contemplated promoters include cell-specific or tissue-specific or developmentally regulated promoters, for example, a promoter that limits the expression of the nucleic acid targeting system to germline or reproductive cells (e.g., promoters of genes encoding DNA ligases, recombinases, replicases, or other genes specifically expressed in germline or reproductive cells). In certain embodiments, the genome alteration is limited only to those cells from which DNA is inherited in subsequent generations, which is advantageous where it is desirable that expression of the genome-editing system be limited in order to avoid genotoxicity or other unwanted effects. All of the patent publications referenced in this paragraph are incorporated herein by reference in their entirety.

Expression vectors or polynucleotides provided herein may contain a DNA segment near the 3′ end of an expression cassette that acts as a signal to terminate transcription and directs polyadenylation of the resultant mRNA and may also support promoter activity. Such a 3′ element is commonly referred to as a “3′-untranslated region” or “3′-UTR” or a “polyadenylation signal.” In some cases, plant gene-based 3′ elements (or terminators) consist of both the 3′-UTR and downstream non-transcribed sequence (Nuccio et al., 2015). Useful 3′ elements include: Agrobacterium tumefaciens nos 3′, tml 3′, tmr 3′, tms 3′, ocs 3′, and tr7 3′ elements disclosed in U.S. Pat. No. 6,090,627, incorporated herein by reference, and 3′ elements from plant genes such as the heat shock protein 17, ubiquitin, and fructose-1,6-biphosphatase genes from wheat (Triticum aestivum), and the glutelin, lactate dehydrogenase, and beta-tubulin genes from rice (Oryza sativa), disclosed in US Patent Application Publication 2002/0192813 A1, incorporated herein by reference.

In certain embodiments, a vector or polynucleotide comprising an expression cassette includes additional components, e.g., a polynucleotide encoding a drug resistance or herbicide gene or a polynucleotide encoding a detectable marker such as green fluorescent protein (GFP) or beta-glucuronidase (gus) to allow convenient screening or selection of cells expressing the vector or polynucleotide. Selectable markers include genes that confer resistance to herbicidal compounds, such as glyphosate, sulfonylureas, glufosinate ammonium, bromoxynil, imidazolinones, and 2,4-dichlorophenoxyacetate (2,4-D). Since the expression of cGRF4-GIF1, cdpGRF4-dpGIF1, or cGmGRF4-GmGIF1 genes can accelerate somatic embryogenesis and embryo maturation, selectable marker genes, selective agents, and conditions can be adjusted to minimize formation of un-edited or untransformed regenerable plant structures (e.g., “escapes”). Such selectable marker genes and selective agents include the HRA gene (Lee et al., 1988, EMBO J 7:1241-1248) which confers resistance to sulfonylureas and imidazolinones, the CP4 gene that confers resistance to glyphosate (US Reissue Patent RE039247, specifically incorporated herein by reference in its entirety and with respect to such genes and related selection methods), the GAT gene which confers resistance to glyphosate (Castle et al., 2004, Science 304:1151-1154), genes that confer resistance to spectinomycin such as the aadA gene (Svab et al., 1990, Plant Mol Biol. 14:197-205) and the bar gene that confers resistance to glufosinate ammonium (White et al., 1990, Nucl. Acids Res. 25:1062), and PAT (or moPAT for corn, see Rasco-Gaunt et al., 2003, Plant Cell Rep. 21:569-76; also see Sivamani et al., 2019) and the PMI gene that permits growth on mannose-containing medium (Negrotto et al., 2000, Plant Cell Rep. 22:684-690).

### Embodiments

The following numbered embodiments also form part of the present disclosure:

1. A composition comprising:
(i) a first recombinant polynucleotide comprising: (a) a polynucleotide encoding a chimeric GRF4-GIF1 (cGRF4-GIF1) polypeptide, optionally wherein the GRF4-GIF1 polypeptide comprises a chimeric dicot plant GRF4-GIF1 (cdpGRF4-dpGIF1) polypeptide or optionally wherein the GRF4-GIF1 polypeptide comprises a chimeric GmGRF4-GmGIF1 (cGmGRF4-GmGIF1) polypeptide comprising from its amino terminus (N-terminus) to its carboxy terminus (C-terminus) a GmGF4 polypeptide comprising an amino acid sequence having at least 92%, 95%, 98%, or 99% sequence identity across the entire length of SEQ ID NO: 1 and a GmGIF1 polypeptide comprising an amino acid sequence having at least 85%, 90%, 95%, 98%, or 99% sequence identity across the entire length of SEQ ID NO: 2 and a terminator which is operably linked to said polynucleotide; and
(ii) a second recombinant polynucleotide comprising a promoter which is operably linked to a polynucleotide encoding an acetohydroxyacid synthase (AHAS) which confers resistance to an AHAS inhibitor and a terminator which is operably linked to said polynucleotide, optionally wherein the first and second recombinant polynucleotides are not covalently linked.

2. The composition of embodiment 1, wherein the GmGF4 polypeptide comprises an amino acid sequence having at least 98% or 99% sequence identity across the entire length of SEQ ID NO: 1 and the GmGIF1 polypeptide comprises an amino acid sequence having at least 98%, or 99% sequence identity across the entire length of SEQ ID NO: 2.

3. The composition of embodiment 1 or 2, wherein the GF4, dpGF4, or GmGF4 polypeptide comprises a QLQ and a WRC domain, optionally wherein QLQ domain comprises the amino acid sequence of SEQ ID NO: 4 and/or wherein the WRC domain comprises the amino acid sequence of SEQ ID NO: 5.

4. The composition of any one of embodiments 1 to 3, wherein the GIF1, dpGIF1, or GmGIF1 polypeptide comprises an SNH domain, optionally wherein the SNH domain comprises the amino acid sequence of SEQ ID NO: 6.

5. The composition of any one of embodiments 1 to 4, wherein the cGmGRF4-GmGIF1 polypeptide comprises an amino acid sequence having at least 95%, 98%, or 99% sequence identity across the entire length of SEQ ID NO: 3.

6. The composition of any one of embodiments 1 to 5, wherein the GF4, dpGF4, or cGmGRF4-GmGIF1 polypeptide encoded by the polynucleotide further comprises a spacer peptide which operably links the C-terminus of said GmGF4 polypeptide to the N-terminus of said GmGIF1 polypeptide, optionally wherein the spacer peptide comprises at least two amino acids comprising alanine, glycine, or comprises a combination of at least two glycine residues and one serine residue.

7. The composition of any one of embodiments 1 to 6, wherein the first recombinant polynucleotide comprises a recombinant DNA molecule or a recombinant RNA molecule.

8. The composition of embodiment 7, wherein the first recombinant DNA molecule further comprises a promoter which is operably linked to the polynucleotide encoding the cGmGRF4-GmGIF1 polypeptide.

9. The composition of any one of embodiments 1 to 8, wherein the second recombinant polynucleotide further comprises an expression cassette comprising a promoter which is operably linked to a polynucleotide encoding an RNA or protein of interest and a terminator which is operably linked to said polynucleotide, optionally wherein the RNA or protein of interest comprise one or more gene editing molecules and/or optionally wherein the second recombinant polynucleotide further comprises one or more DNA elements which provide for bacterially mediated transfection or transformation of a plant cell.

10. The composition of embodiment 9, wherein the polynucleotide encodes an RNA comprising a guide RNA (gRNA) which is directed to a target DNA sequence found in a soybean genome and optionally wherein the target DNA sequence is exclusively found in a soybean genome.

11. A bacterial cell comprising the composition of any one of embodiments 1 to 10, optionally wherein the bacterial cell is an Agrobacterium sp., Rhizobium sp., Sinorhizobium sp., Mesorhizobium sp., Bradyrhizobium sp., Azobacter sp., or Phyllobacterium sp. cell.

12. A soybean plant cell comprising the composition of any one of embodiments 1 to 10.

13. The soybean plant cell of embodiment 12, wherein the second recombinant polynucleotide is stably incorporated into the genome of the soybean plant cell.

14. The soybean plant cell of embodiment 11 or 12, wherein the first recombinant polynucleotide is transiently expressed in the soybean plant cell and/or is not stably incorporated into the genome of the soybean plant cell.

15. The soybean plant cell of any one of embodiments 12 to 14, wherein the soybean plant cell comprises elite soybean germplasm and/or soybean germplasm which is recalcitrant to regeneration.

16. The soybean plant cell of any one of embodiments 12 to 15, wherein expression of the chimeric polypeptide increases the regeneration capacity of the soybean plant cell in comparison to a control soybean plant cell lacking the chimeric polypeptide.

17. The soybean plant cell of any one of embodiments 12 to 16, wherein the percentage of transgenic or genome edited shoots recovered from the soybean plant cell in a transformation or genome editing procedure is increased by up to about 9-fold in comparison to a control soybean plant cell lacking the chimeric polypeptide.

18. The soybean plant cell of any one of embodiments 12 to 17, further comprising a genome editing system.

19. The soybean plant cell of embodiment 18, wherein the genome editing system comprises a CRISPR-based system, a transcription activator-like effector nuclease (TALEN) system, or a zinc finger nuclease (ZFN) system, and optionally a donor DNA template polynucleotide.

20. The soybean plant cell of embodiment 19, wherein the CRISPR-based system comprises (i) an RNA-guided nuclease or a polynucleotide encoding the RNA-guided nuclease; and (ii) a guide RNA or a polynucleotide encoding the gRNA, optionally wherein the gRNA is directed to a target DNA sequence which is exclusively found in a soybean genome.

21. A soybean plant, tissue, organ, callus, or cell culture comprising the soybean plant cell of any one of embodiments 12 to 20.

22. A method of producing a regenerable plant structure, the method comprising: culturing the soybean plant cell of any one of embodiments 12 to 20 in the presence of an acetohydroxyacid synthase inhibitor at a concentration sufficient to select for a regenerable plant structure comprising the second recombinant polynucleotide, thereby producing the regenerable plant structure.

23. The method of embodiment 22, wherein the first and/or second recombinant polynucleotide is operably linked to a heterologous promoter functional in a plant cell.

24. The method of embodiment 22 or 23, wherein the second recombinant polynucleotide is stably incorporated into the genome of the soybean plant cell.

25. The method of any one of embodiments 22 to 24, wherein the first recombinant polynucleotide is transiently expressed in the soybean plant cell and/or is not stably incorporated into the genome of the soybean plant cell.

26. The method of any one of embodiments 22 to 25, wherein the soybean plant cell comprises a regeneration-recalcitrant germplasm.

27. The method of any one of embodiments 22 to 26, wherein the regenerable plant structure comprises a somatic embryo, embryogenic callus, somatic meristem, organogenic callus, a shoot, or a shoot further comprising roots.

28. The method of any one of embodiments 22 to 27, further comprising introducing the composition of any one of embodiments 1 to 10 into a soybean plant cell to obtain the soybean plant cell of any one of embodiments 12 to 20.

29. The method of embodiment 28, wherein the introducing comprises bacterial-mediated transformation or biolistic-mediated transformation.

30. The method of any one of embodiments 22 to 29, wherein expression of the polypeptide results an increase in the percentage of transgenic or genome edited shoots recovered from the soybean plant cell in a transformation or genome editing procedure by up to about 9-fold in comparison to a control soybean plant cell lacking the chimeric polypeptide.

31. The method of any one of embodiments 22 to 30, further comprising introducing a genome editing system into the soybean plant cell.

32. The method of embodiment 31, wherein the genome editing system comprises a CRISPR-based system, a transcription activator-like effector nuclease (TALEN) system, or a zinc finger nuclease (ZFN) system, and optionally a donor template polynucleotide.

33. The method of embodiment 32, wherein the CRISPR-based system comprises (i) an RNA-guided nuclease or a polynucleotide encoding the RNA-guided nuclease; (ii) a guide RNA or a polynucleotide encoding the gRNA; and optionally (ii) a donor.

34. The method of any one of embodiments 22 to 33, wherein the regenerable structure comprises a shoot of 1 to 2, 3, or 4 centimeters (cm) in length.

35. The method of embodiment 34, wherein the culturing further comprises transferring the shoot to a rooting media.

36. The method of embodiment 35, further comprising obtaining a plantlet comprising a shoot and roots from the shoot which was transferred to the rooting media.

36. The method of any one of embodiments 22 to 36, where the AHAS inhibitor comprises a sulfonylurea, imidazolinone, triazoloyrimidine, or triazolinone herbicide.

37. The method of embodiment 36, wherein the sulfonylurea herbicide is bensulfuron-methyl, chlorsulfuron, ethametsulfuron-methyl, foramsulfuron, halosulfuron, mesosulfuron-WO methyl, metsulfuron-methyl, nicosulfuron, oxasulfuron, primisulfuron-methyl, prosulfuron, rimsulfuron, sulfometuron-methyl, sulfosulfuron, thifensulfuron-methyl, triasulfuron, or triflusulfuron-methyl.

38. The method of embodiment 36, wherein the imidazolinone herbicide is imazapyr, imazapic, imazethapyr, imazamox, imazamethabenz, or imazaquin.

### EXAMPLES

### Example 1

This example describes the methods used in the experiments of Example 2.

### Vectors

Binary T-DNA vectors with the following expression cassettes between left and right borders were used in the experiments. pIN-2361 and pIN-2289 comprised expression cassettes that encode the gmGRF4-gmGIF1 chimera set forth in SEQ ID NO: 3.

TABLE 1
Vector Description

| ID | Constructs |
|---|---|
| pIN-2361 | AtUbi10_p:Gm_opt_EPSPS:Pea_rbcs_t::SlUbi10p:Cas:HSPt::AtU6- |
| | 26p:gRNA:polIIIt::PcUBIp:GRF4-GIF1:NOSt |
| pIN-2289 | AtUbi10_p:AtAHAS:Pea_rbcs_t::SlUbi10p:Cas:HSPt::AtU6- |
| | 26p:gRNA:polIIIt::PcUBIp:GRF4-GIF1:NOSt |
| pIN1304 | AtUbi10_p:Gm_opt_EPSPS:Pea_rbcs_t::SlUbi10p:Cas:HSPt::AtU6- |
| | 26p:gRNA:polIIIt |
| pIN2291 | AtUbi10_p:AtAHAS:Pea_rbcs_t::SlUbi10p:Cas:HSPt::AtU6-26p:gRNA:polIIIt |

p = promoter
t = 3′UTR and terminator

### Example 2. Transformation Experiments

The soybean lines used in transformation experiments were NINF1170, which is relatively easy to transform, and TEND1593 and TENE 1686, which are relatively recalcitrant to transformation.

Transgenic TO soybean events were made by Agrobacterium-mediated transformation with vectors pIN-2361, pIN-2289 (with gmGRF4-gmGIF1 chimera-containing expression cassettes) and pIN-1304, pIN-2291 (Controls, without gmGRF4-gmGIF1 chimera-containing expression cassettes). Sterilized soybean seeds were imbibed in water overnight, and explants were prepared as mature cotyledon halves with trimmed hypocotyls. The explants went through the typical transformation and regeneration steps of infection and co-cultivation, shoot induction and elongation and selection, rooting, and transplanting to soil to produce Tl seeds (see, for example, Li et al, Optimization of Agrobacterium-Mediated Transformation in Soybean (2017) Front. Plant Sci., DOI: 10.3389/fpls.2017.00246) with the exception that some shoots were transferred to rooting media at a length of about 1.5 cm (“1.5 cm” in Treatment Column of Table 2) rather than the customary shoot length of 5 cm (“5 cm” in the Treatment Column of Table 2).

TABLE 2
Transformation Experiment Results with gmGRF4-gmGIF1 chimera vectors and control vectors

| | | | | | Starting | # | Percent |
|---|---|---|---|---|---|---|---|
| | | | | | # | transgenic | transgenic |
| Experiment # | Treatment | Genotype | Plasmid | Selection | explants | shoots | shoots |
| GMIW000317 | Chimera_5 cm | NINF1170 | pIN2361 | Glyphosate | 315 | 8 | 2.5 |
| GMIW000317 | Chimera_1.5 cm | NINF1170 | pIN2361 | Glyphosate | 309 | 4 | 1.3 |
| GMIW000317 | Control_5 cm | NINF1170 | pIN1304 | Glyphosate | 325 | 9 | 2.8 |
| GMIW000318 | Chimera_5 cm | NINF1170 | pIN2289 | Imazapyr | 161 | 3 | 1.9 |
| GMIW000318 | Chimera_1.5 cm | NINF1170 | pIN2289 | Imazapyr | 159 | 11 | 6.9 |
| GMIW000318 | Control_5 cm | NINF1170 | pIN2291 | Imazapyr | 167 | 7 | 4.2 |
| GMIW000319 | Chimera_5 cm | TEND1593 | pIN2289 | Imazapyr | 291 | 3 | 1.0 |
| GMIW000319 | Chimera_1.5 cm | TEND1593 | pIN2289 | Imazapyr | 267 | 9 | 3.4 |
| GMIW000319 | Control_5 cm | TEND1593 | pIN2291 | Imazapyr | 280 | 1 | 0.4 |
| GMIW000320 | Chimera_5 cm | TENE1686 | pIN2289 | Imazapyr | 282 | 26 | 9.2 |
| GMIW000320 | Chimera_1.5 cm | TENE1686 | pIN2289 | Imazapyr | 276 | 14 | 5.1 |
| GMIW000320 | Control_5 cm | TENE1686 | pIN2291 | Imazapyr | 264 | 3 | 1.1 |
| GMIW000321 | Chimera_5 cm | NINF1170 | pIN2289 | Imazapyr | 147 | 1 | 0.7 |
| GMIW000321 | Chimera_1.5 cm | NINF1170 | pIN2289 | Imazapyr | 160 | 6 | 3.8 |
| GMIW000321 | Control_5 cm | NINF1170 | pIN2291 | Imazapyr | 158 | 0 | 0.0 |

Genotypes TEND1593 and TENE1686 were tested earlier for transformation in our transformation platform in experiments that resulted in very low transformation efficiency. In the present experiment disclosed here, these genotypes transformed with control plasmid (“Control” in the Treatment column of Table 2) still showed very low transformation efficiency. However, when the genotypes were transformed using the chimeric GmGRF4-GmGIF1 fusion (SEQ ID NO: 3; “Chimera” in the Treatment column), a significant boost in transformation efficiency was observed. TEND1593 showed 0.4% TE with control vector while it showed 1% TE with the regular protocol (with GmGRF4-GmGIF1 fusion) where shoots were harvested at a height of 3-4 cm and 3.4% TE (with GmGRF4-GmGIF1 fusion) when the regenerating shoots were harvested early on when they were about 1.5 cm only. Similarly, genotype TENE1686 showed 1.1% TE with control vector (no GmGRF4-GmGIF1 fusion) while it showed 9.2% TE with the regular protocol (with GmGRF4-GmGIF1 fusion) where shoots were harvested when they reach a height of 3-4 cm and 5.1% TE (with GmGRF4-GmGIF1 fusion) when the regenerating shoots were harvested early on when they were about 1.5 cm only.

The breadth and scope of the present disclosure should not be limited by any of the above-described examples.

### Sequences

>GMGRF4
(SEQ ID NO: 1)
MNISGGGGTVMGFSSNGRSPFTVSQWQELEHQALIFKYMVAGLPV
PPDLVLPIQKSFDSTLSHAFFHHPTLSYCSFYGKKVDPEPGRCRR
TDGKKWRCSKEAYPDSKYCERHMHRGRNRSRKPVESQTMTHSSST
VTSLTVTGGGDSNGTVNFQNLPTNAFGNLQGTDSGTDRTNYHLDS
IPYAIPSKEYRCLQGLKSEGGEHCFFSEASGSNKVLQMESQLENT
WPSMSTRVASFSTSKSSTDSLLHSDYPQHSFLSGEYASGEHVKEE
GQPLRPFSNEWPKSRESWSGLEDDISNQTAFSTTQLSISIPMSSD
FSATSSQSPHGENEIQFR
QLQ domain underlined; WRC domain in bold
GMGIF1
(SEQ ID NO: 2)
MQQHLMQMQPMMAAYYPNNVTTDHIQQYLDENKSLILKIVESQNS
GKLSECAENQARLQRNLMYLAAIADSQPQPPTMSGQYPPSGMMQQ
GAQYMQAQQQAQQMTPQQLMAARSSLLYAQQPYSALQQQQAMHSA
LGSSSGLHMLQSEGSNVNVGGGFPDFVRGGSSTGEGLHSGGRGII
GSSKQEMGGSSEGRGEGGENLYLKVADDGN
SNH domain underlined and in bold
GMGRF4-GMGIF1 CHIMERA
(SEQ ID NO: 3)
MNISGGGGTVMGFSSNGRSPFTVSQWQELEHQALIFKYMVAGLPV
PPDLVLPIQKSFDSTLSHAFFHHPTLSYCSFYGKKVDPEPGRCRR
TDGKKWRCSKEAYPDSKYCERHMHRGRNRSRKPVESQTMTHSSST
VTSLTVTGGGDSNGTVNFQNLPTNAFGNLQGTDSGTDRTNYHLDS
IPYAIPSKEYRCLQGLKSEGGEHCFFSEASGSNKVLQMESQLENT
WPSMSTRVASFSTSKSSTDSLLHSDYPQHSFLSGEYASGEHVKEE
GQPLRPFSNEWPKSRESWSGLEDDISNQTAFSTTQLSISIPMSSD
FSATSSQSPHGENEIQFRAAAAMQQHLMQMQPMMAAYYPNNVTTD
HIQQYLDENKSLILKIVESQNSGKLSECAENQARLQRNLMYLAAI
ADSQPQPPTMSGQYPPSGMMQQGAQYMQAQQQAQQMTPQQLMAAR
SSLLYAQQPYSALQQQQAMHSALGSSSGLHMLQSEGSNVNVGGGF
PDFVRGGSSTGEGLHSGGRGIIGSSKQEMGGSSEGRGEGGENLYL
KVADDGN
QLQ DOMAIN
(SEQ ID NO: 4)
PFTVSQWQELEHQALIFKYMVAGLPVPPDLVLPIQ
WRC DOMAIN
(SEQ ID NO: 5)
DPEPGRCRRTDGKKWRCSKEAYPDSKYCERHMHRGRNRSRKPVE
SNH DOMAIN
(SEQ ID NO: 6)
NNVTTDHIQQYLDENKSLILKIVESQNSGKLSECAENQARLQRNL
MYLAAIADSQPQPPT

What is claimed is:

1. A composition comprising:
(i) a first recombinant polynucleotide comprising: (a) polynucleotide encoding a chimeric GRF4-GIF1 (cGRF4-GIF1) polypeptide, optionally wherein the GRF4-GIF1 polypeptide comprises a chimeric dicot plant GRF4-GIF1 (cdpGRF4-dpGIF1) polypeptide or a chimeric GmGRF4-GmGIF1 (cGmGRF4-GmGIF1) polypeptide, said cGmGRF4-GmGIF1 polypeptide comprising from its amino terminus (N-terminus) to its carboxy terminus (C-terminus) a GmGF4 polypeptide comprising an amino acid sequence having at least 92%, 95%, 98%, or 99% sequence identity across the entire length of SEQ ID NO: 1 and a GmGIF1 polypeptide comprising an amino acid sequence having at least 85%, 90%, 95%, 98%, or 99% sequence identity across the entire length of SEQ ID NO: 2; and (b) a terminator which is operably linked to said polynucleotide; and
(ii) a second recombinant polynucleotide comprising a promoter which is operably linked to a polynucleotide encoding an acetohydroxyacid synthase (AHAS) which confers resistance to an AHAS inhibitor and a terminator which is operably linked to said polynucleotide.

2. The composition of claim 1, wherein the GmGF4 polypeptide comprises an amino acid sequence having at least 98% or 99% sequence identity across the entire length of SEQ ID NO: 1 and the GmGIF1 polypeptide comprises an amino acid sequence having at least 98%, or 99% sequence identity across the entire length of SEQ ID NO: 2.

3. The composition of claim 1, wherein the GF4, dpGF4, or GmGF4 polypeptide comprises a QLQ and a WRC domain, optionally wherein QLQ domain comprises the amino acid sequence of SEQ ID NO: 4 and/or wherein the WRC domain comprises the amino acid sequence of SEQ ID NO: 5.

4. The composition of claim 1, wherein the GIF1, dpGIF1, or GmGIF1 polypeptide comprises an SNH domain, optionally wherein the SNH domain comprises the amino acid sequence of SEQ ID NO: 6.

5. The composition of claim 1, wherein the cGmGRF4-GmGIF1 polypeptide comprises an amino acid sequence having at least 95%, 98%, or 99% sequence identity across the entire length of SEQ ID NO: 3.

6. The composition of claim 1, wherein the GF4, dpGF4, or cGmGRF4-GmGIF1 polypeptide encoded by the polynucleotide further comprises a spacer peptide which operably links the C-terminus of said GmGF4 polypeptide to the N-terminus of said GmGIF1 polypeptide, optionally wherein the spacer peptide comprises at least two amino acids comprising alanine, glycine, or comprises a combination of at least two glycine residues and one serine residue.

7. The composition of claim 1, wherein the first recombinant polynucleotide comprises a recombinant DNA molecule or a recombinant RNA molecule.

8. The composition of claim 1, wherein the recombinant DNA molecule further comprises a promoter which is operably linked to the polynucleotide encoding the cGmGRF4-GmGIF1 polypeptide.

9. The composition of claim 1, wherein the second recombinant polynucleotide further comprises an expression cassette comprising a promoter which is operably linked to a polynucleotide encoding an RNA or protein of interest and a terminator which is operably linked to said polynucleotide, optionally wherein the RNA or protein of interest comprise one or more gene editing molecules and/or optionally wherein the second recombinant polynucleotide further comprises one or more DNA elements which provide for bacterially mediated transfection or transformation of a plant cell.

10. The composition of claim 9, wherein the polynucleotide encodes an RNA comprising a guide RNA (gRNA) which is directed to a target DNA sequence found in a soybean genome and optionally wherein the target DNA sequence is exclusively found in a soybean genome.

11. A bacterial cell comprising the composition of any one of claims 1 to 10, optionally wherein the bacterial cell is an Agrobacterium sp., Rhizobium sp., Sinorhizobium sp., Mesorhizobium sp., Bradyrhizobium sp., Azobacter sp., or Phyllobacterium sp. cell.

12. A soybean plant cell comprising the composition of any one of claims 1 to 10.

13. The soybean plant cell of claim 12, wherein the second recombinant polynucleotide is stably incorporated into the genome of the soybean plant cell.

14. The soybean plant cell of claim 12, wherein the first recombinant polynucleotide is transiently expressed in the soybean plant cell and/or is not stably incorporated into the genome of the soybean plant cell.

15. The soybean plant cell of claim 12, wherein the soybean plant cell comprises elite soybean germplasm and/or soybean germplasm which is recalcitrant to regeneration.

16. The soybean plant cell of claim 12, wherein expression of the chimeric polypeptide increases the regeneration capacity of the soybean plant cell in comparison to a control soybean plant cell lacking the chimeric polypeptide.

17. The soybean plant cell of claim 12, wherein the percentage of transgenic or genome edited shoots recovered from the soybean plant cell in a transformation or genome editing procedure is increased by up to about 9-fold in comparison to a control soybean plant cell lacking the chimeric polypeptide.

18. The soybean plant cell of claim 12, further comprising a genome editing system.

19. The soybean plant cell of claim 18, wherein the genome editing system comprises a CRISPR-based system, a transcription activator-like effector nuclease (TALEN) system, or a zinc finger nuclease (ZFN) system, and optionally a donor DNA template polynucleotide.

20. The soybean plant cell of claim 19, wherein the CRISPR-based system comprises (i) an RNA-guided nuclease or a polynucleotide encoding the RNA-guided nuclease; and (ii) a guide RNA or a polynucleotide encoding the gRNA, optionally wherein the gRNA is directed to a target DNA sequence which is exclusively found in a soybean genome.

21. A soybean plant, tissue, organ, callus, or cell culture comprising the soybean plant cell of claim 12.

22. A method of producing a regenerable plant structure, the method comprising:
culturing the soybean plant cell of claim 12 in the presence of an acetohydroxyacid synthase (AHAS) inhibitor at a concentration sufficient to select for a regenerable plant structure comprising the second recombinant polynucleotide, thereby producing the regenerable plant structure.

23. The method of claim 22, wherein the first and/or second recombinant polynucleotide is operably linked to a heterologous promoter functional in a plant cell.

24. The method of claim 22, wherein the second recombinant polynucleotide is stably incorporated into the genome of the soybean plant cell.

25. The method of claim 22, wherein the first recombinant polynucleotide is transiently expressed in the soybean plant cell and/or is not stably incorporated into the genome of the soybean plant cell.

26. The method of claim 22, wherein the soybean plant cell comprises a regeneration-recalcitrant germplasm.

27. The method of claim 22, wherein the regenerable plant structure comprises a somatic embryo, embryogenic callus, somatic meristem, organogenic callus, a shoot, or a shoot further comprising roots.

28. The method of claim 22, further comprising introducing the composition comprising the first recombinant polynucleotide and the second recombinant polynucleotide into a soybean plant cell to obtain the soybean plant cell of claim 12 that is cultured.

29. The method of claim 28, wherein the introducing comprises bacterial-mediated transformation or biolistic-mediated transformation.

30. The method of claim 22, wherein expression of the polypeptide results an increase in the percentage of transgenic or genome edited shoots recovered from the soybean plant cell in a transformation or genome editing procedure by up to about 9-fold in comparison to a control soybean plant cell lacking the chimeric polypeptide.

31. The method of claim 22, further comprising introducing a genome editing system into the soybean plant cell.

32. The method of claim 31, wherein the genome editing system comprises a CRISPR-based system, a transcription activator-like effector nuclease (TALEN) system, or a zinc finger nuclease (ZFN) system, and optionally a donor template polynucleotide.

33. The method of claim 32, wherein the CRISPR-based system comprises (i) an RNA-guided nuclease or a polynucleotide encoding the RNA-guided nuclease; (ii) a guide RNA or a polynucleotide encoding the gRNA; and optionally (ii) a donor.

34. The method of claim 22, wherein the regenerable structure comprises a shoot of 1 to 2, 3, or 4 centimeters (cm) in length.

35. The method of claim 34, wherein the culturing further comprises transferring the shoot to a rooting media.

36. The method of claim 34, further comprising obtaining a plantlet comprising a shoot and roots from the shoot which was transferred to the rooting media.

36. The method of claim 22, where the AHAS inhibitor comprises a sulfonylurea, imidazolinone, triazoloyrimidine, or triazolinone herbicide.

37. The method of claim 36, wherein the sulfonylurea herbicide is bensulfuron-methyl, chlorsulfuron, ethametsulfuron-methyl, foramsulfuron, halosulfuron, mesosulfuron-methyl, metsulfuron-methyl, nicosulfuron, oxasulfuron, primisulfuron-methyl, prosulfuron, rimsulfuron, sulfometuron-methyl, sulfosulfuron, thifensulfuron-methyl, triasulfuron, or triflusulfuron-methyl.

38. The method of claim 36, wherein the imidazolinone herbicide is imazapyr, imazapic, imazethapyr, imazamox, imazamethabenz, or imazaquin.


SEQUENCE LISTING (15 sequences)

SEQ ID NO: 1 - protein, 333 aa, Glycine max
MNISGGGGTVMGFSSNGRSPFTVSQWQELEHQALIFKYMVAGLPVPPDLVLPIQKSFDSTLSHAFFHHPTLSYCSFYGKKVDPEPGRCRRTDGKKWRCSKEAYPDSKYCERHMHRGRNRSRKPVESQTMTHSSSTVTSLTVTGGGDSNGTVNFQNLPTNAFGNLQGTDSGTDRTNYHLDSIPYAIPSKEYRCLQGLKSEGGEHCFFSEASGSNKVLQMESQLENTWPSMSTRVASFSTSKSSTDSLLHSDYPQHSFLSGEYASGEHVKEEGQPLRPFSNEWPKSRESWSGLEDDISNQTAFSTTQLSISIPMSSDFSATSSQSPHGENEIQFR

SEQ ID NO: 2 - protein, 210 aa, Glycine max
MQQHLMQMQPMMAAYYPNNVTTDHIQQYLDENKSLILKIVESQNSGKLSECAENQARLQRNLMYLAAIADSQPQPPTMSGQYPPSGMMQQGAQYMQAQQQAQQMTPQQLMAARSSLLYAQQPYSALQQQQAMHSALGSSSGLHMLQSEGSNVNVGGGFPDFVRGGSSTGEGLHSGGRGIIGSSKQEMGGSSEGRGEGGENLYLKVADDGN

SEQ ID NO: 3 - protein, 547 aa, synthetic construct
MNISGGGGTVMGFSSNGRSPFTVSQWQELEHQALIFKYMVAGLPVPPDLVLPIQKSFDSTLSHAFFHHPTLSYCSFYGKKVDPEPGRCRRTDGKKWRCSKEAYPDSKYCERHMHRGRNRSRKPVESQTMTHSSSTVTSLTVTGGGDSNGTVNFQNLPTNAFGNLQGTDSGTDRTNYHLDSIPYAIPSKEYRCLQGLKSEGGEHCFFSEASGSNKVLQMESQLENTWPSMSTRVASFSTSKSSTDSLLHSDYPQHSFLSGEYASGEHVKEEGQPLRPFSNEWPKSRESWSGLEDDISNQTAFSTTQLSISIPMSSDFSATSSQSPHGENEIQFRAAAAMQQHLMQMQPMMAAYYPNNVTTDHIQQYLDENKSLILKIVESQNSGKLSECAENQARLQRNLMYLAAIADSQPQPPTMSGQYPPSGMMQQGAQYMQAQQQAQQMTPQQLMAARSSLLYAQQPYSALQQQQAMHSALGSSSGLHMLQSEGSNVNVGGGFPDFVRGGSSTGEGLHSGGRGIIGSSKQEMGGSSEGRGEGGENLYLKVADDGN

SEQ ID NO: 4 - protein, 35 aa, Glycine max
PFTVSQWQELEHQALIFKYMVAGLPVPPDLVLPIQ

SEQ ID NO: 5 - protein, 44 aa, Glycine max
DPEPGRCRRTDGKKWRCSKEAYPDSKYCERHMHRGRNRSRKPVE

SEQ ID NO: 6 - protein, 60 aa, Glycine max
NNVTTDHIQQYLDENKSLILKIVESQNSGKLSECAENQARLQRNLMYLAAIADSQPQPPT

SEQ ID NO: 7 - protein, 5 aa, synthetic construct
GSGGS

SEQ ID NO: 8 - protein, 6 aa, synthetic construct
GGSGGS

SEQ ID NO: 9 - protein, 4 aa, synthetic construct
GGGS

SEQ ID NO: 10 - protein, 4 aa, synthetic construct
GGSG

SEQ ID NO: 11 - protein, 5 aa, synthetic construct
GGSGG

SEQ ID NO: 12 - protein, 5 aa, synthetic construct
GSGSG

SEQ ID NO: 13 - protein, 5 aa, synthetic construct
GSGGG

SEQ ID NO: 14 - protein, 5 aa, synthetic construct
GGGSG

SEQ ID NO: 15 - protein, 5 aa, synthetic construct
GSSSG